(19) United States
(12) Patent Application Publication
(10) Pub. No.: US 2003/0153051 A1
(43) Pub. Date: Aug. 14, 2003

(54) Cholesterol oxidase

(76) Inventors: Rikizo Aono, Yokohama-shi (JP); Toru Hamaya, Sakado-Shi (JP); Toshiaki Kono, Sakado-Shi (JP)

Correspondence Address: WENDEROTH, LIND & PONACK, L.L.P., 2033 K STREET N. W., SUITE 800, WASHINGTON, DC, 20006-1021, US

(21) Appl. No.: 10/377,820

(22) Filed: Mar. 4, 2003

Related U.S. Application Data
(63) Continuation of application No. 09/623,219, filed on Aug. 30, 2000.
Application No. 09/623,219 is a 371 of international application No. PCT/JP99/01022, filed on Mar. 3, 1999.

(30) Foreign Application Priority Data
Mar. 3, 1998 (JP) 50855/1998

(51) Int. Cl. C12N 9/00; C12P 21/04

(52) U.S. Cl. 435/70.1; 435/52; 435/183

(57) ABSTRACT

A novel cholesterol oxidase is disclosed. The enzyme according to the present invention has the following properties: acts on cholesterol to convert it to cholest-5-en-3-one; acts 3β-sterols but not on 3α-hydroxysteroids; optimum pH: 5.0-8.5; and stable pH: 4 to 11. This enzyme carries out the oxidation reaction at high velocity at low substrate concentrations, reacts in a broad pH range, is heat resistant, and reacts at high reaction velocity even in the presence of an organic solvent. The enzyme according to the present invention is useful as a reagent for measuring cholesterol concentration, as a composition for the extermination of harmful insects, and as a bleaching agent.

### BACKGROUND OF THE INVENTION

1. Field of the Invention

The present invention relates to cholesterol oxidase which can be used for the measurement of cholesterol concentration in body fluids and food products, and for the production of cholesterol derivatives, pesticides, detergents and the like.

2. Background Art

Cholesterol oxidase is an oxidation enzyme that catalyzes the reaction between a 3β-hydroxysteroid and oxygen to produce the corresponding 3-oxosteroid and hydrogen peroxide. To date, this enzyme has been developed and studied for the measurement of cholesterol concentrations in body fluids (Japanese Patent Laid-open No. 6-169765, etc.), for the production of cholesterol derivatives (Japanese Patent Laid-open No. 6-113883, etc.), and for use in pesticides (Purcell J. P. et al., Biochem. Biophy. Res. Comm., 196, 3, 1406-1413 (1993); U.S. Pat. No. 5,558,862, etc.), detergents (WO89/09813, etc.) and the like.

This enzyme is known to be produced by various microorganisms, such as Streptomyces (Japanese Patent Laid-open No. 62-285789), Brevibacterium (Japanese Patent Laid Open No. 4-218367), Rhodococcus (Japanese Patent Publication 3-503478), Pseudomonas (Japanese Patent Laid-open 6-189754), or the like.

Properties required of this enzyme differ depending on its use. For example, heat stability is required for the measurement of cholesterol concentration in body fluids; therefore a search for novel enzymes (Japanese Patent Laid-open No. 6-169765) and a modification of microorganisms using protein engineering (Japanese Patent Laid-open No. 8-242860) have been attempted to attain such property. Further, resistance to organic solvents is required for the production of cholesterol derivatives.

In addition to the abovementioned properties required for individual uses in many uses including the measurement of cholesterol in body fluids and foods, it is preferable for the enzyme to catalyze the oxidation of cholesterol at high velocity at low concentrations of the substrate (cholesterol). Therefore, the development of cholesterol oxidase that is excellent in this respect was needed.

In this connection, it is described in Applied and Environmental Microbiology, July 1994, 2518-2523 that a strain of genus Pseudomonas has cholesterol oxidation activity.

### SUMMARY OF THE INVENTION

The present inventors recently found cholesterol oxidase produced by a strain of Pseudomonas ST-200 and successfully isolated and purified the enzyme. This enzyme can carry out the oxidation reaction at high velocity in the presence of a low concentration of substrates, can act in a broad pH range, is heat-resistant, and furthermore, is activated strongly by organic solvents. The present invention is based on the finding.

The enzyme according to the present invention is cholesterol oxidase produced by ST-200 strain (FERM BP-6661).

The enzyme according to the present invention has the following properties:

(1) Function: acting on cholesterol to convert it to cholest-5-en-3-one; acting on cholest-5-en-3-one to convert it to 6β-perhydroxycholest-4-en-3-one;

(2) Substrate specificity: acts on 3β-sterols and not on 3α-hydroxysteroid;

(3) Optimum pH: 5.0 to 8.5; and

(4) Stable pH: 4 to 11.

The enzyme according to the present invention is useful as a reagent for measuring cholesterol concentration, as a composition for exterminating pests, and as a bleaching agent.

Deposition of Microorganisms

Pseudomonas sp. ST-200 strain was deposited at the National Institute of Bioscience and Human-Technology Agency of Industrial Science and Technology (1-3 Higashi 1-Chome, Tsukuba City, Ibaraki Prefecture, Japan), dated Feb. 4, 1998. The access number is FERM BP-6661.

### BRIEF DESCRIPTION OF THE DRAWINGS

FIG. 1 shows the pH dependence of the enzyme according to the present invention. Buffer solutions used are ◯: 50 mM acetic acid—sodium acetate buffer; ▴: 50 mM monopotassium phosphate—disodium phosphate buffer; □: 50 mM tris—hydrochloric acid buffer; and ●: 50 mM sodium carbonate—sodium hydrogencarbonate buffer.

FIG. 2 shows the temperature dependence of the enzyme according to the present invention.

FIG. 3 shows the pH stability of the enzyme according to the present invention. Buffers used are ◯: 50 mM acetic acid—sodium acetate buffer; ▴: 50 mM monopotassium phosphate—disodium phosphate buffer; □: 50 mM tris—hydrochloric acid buffer; ●: 50 mM sodium carbonate—sodium hydrogencarbonate buffer; and ▪: 50 mM sodium chloride—sodium hydroxide buffer.

FIG. 4 shows the temperature stability of the enzyme according to the present invention.

### DETAILED DESCRIPTION OF THE INVENTION

Properties of Enzyme

The enzyme according to the present invention is a cholesterol oxidase that can act on cholesterol to oxidize it. More specifically, the enzyme can convert cholesterol to cholest-5-en-3-one, and cholest-5-en-3-one to 6β-perhydroxycholest-4-en-3-one.

Upon oxidation of cholesterol, the enzyme is transformed into a reduced-form enzyme as a result of the reduction of the coenzyme flavin. This reduced-form enzyme adds an oxygen molecule, for example, oxygen that can be found in the air, onto cholest-5-en-3-one to yield 6β-perhydroxycholest-4-en-3-one. Then, the dissociated-form cholesterol oxidase reduces oxygen to yield hydrogen peroxide. As a result, the reduced-form enzyme is oxidized into the oxidized-form enzyme. This oxidized-form enzyme can again acts on cholesterol to oxidize it. The enzyme according to the present invention includes both oxidized-form enzyme and reduced-form enzyme.

The enzyme according to the present invention has substrate specificity for3β-sterols. The term “3β-sterols” as used herein means sterols having a hydroxy group at position 3 in the β configuration, including cholesterol, β-sitosterol, β-cholestanol, β-stigmasterol, pregnenolone, ergosterol, dehydroepiandrosterone and epiandrosterone. The enzyme according to the present invention does not act on 3α-hydroxysteroids, such as epicholesterol.

The enzyme according to the present invention has an optimum pH range of 5.0 to 8.5 for its activity. The enzyme is stable at a pH range of 4.0 to 11.

In the reaction of the cholesterol oxidase according to the present invention with cholesterol, a ratio of a maximum reaction velocity (Vₘₐₓ: μmole·min⁻¹·mg⁻¹) to Michaelis constant (Kₘ: μM), i.e., Vₘₐₓ/Kₘ, is 0.23 in the presence of 0.3% surfactant Triton X-100, and the Vₘₐₓ/Kₘ is 3.2 in the presence of 0.03% Triton X-100. Cholesterol oxidase having such high Vₘₐₛ/Kₘ values has not been reported yet.

The enzyme according to the present invention has an optimum temperature of approximately 60° C. and is stable at temperatures between 4° C. and 55° C. Known natural cholesterol oxidases have low heat stability. In contrast, the enzyme according to the present invention has a broad range of temperature stability.

The reaction velocity of the cholesterol oxidase described in the present invention is increased by organic solvents having a logP_{ow} between 2.1 and 4.5, such as benzene (2.1), toluene (2.6), paraxylene (3.1), propylbenzene (3.7), diphenylmethane (4.2), cyclooctane (4.5) (figures in the parentheses indicate logP_{ow} values), and the like. Therefore, the enzyme according to the present invention can convert 3β-sterols at high efficiency in an organic solvent. logP_{ow} values are common logarithms of P_{ow} which is the partition constant of given substances between two phases, i.e., water and n-octanol, and they indicate the polarity of the substances. For a given substance, the value P_{ow} is calculated to be (concentration in n-octanol phase)/(concentration in water phase). Therefore, substances having low logPow values have a high polarity. The logP_{ow} values defined in the present invention for various organic solvents are calculated from their structures. The calculation can be carried out using the method described in Chemical Review (Leo, A. J., Calculating logP_{oct} from structure. 93; 1281-1306), the logP_{ow} calculation program C logP ver.1.0.3 (Bio-Byte Corp., California), or the like.

The enzyme according to the invention has a molecular weight of about 60 kDa as measured by SDS polyacrylamide gel electrophoresis.

Enzyme activity of the enzyme according to the present invention is inhibited by silver nitrate and mercury chloride when cholesterol is used as a substrate.

Production of Enzyme

The enzyme according to the present invention can be produced by culturing Pseudomonas ST-200 and isolating and purifying the resulting culture product. The culture method is not restricted and any liquid culture or solid culture can be used. A medium containing an appropriate carbon source and nitrogen source and, if necessary, an appropriate amount of phosphates, inorganic ions or the like, can be used. It is preferable to appropriately control conditions for stirring and aeration during the culture. Furthermore, contamination by other microorganisms can be prevented without reducing the yield of cholesterol oxidase production by loading cyclohexane during the culture, since Pseudomonas ST-200 is resistant to cyclohexane.

Most of the known cholesterol oxidases are produced intracellularly in microorganisms and a process for decomposing cells is essential for their extraction, which requires a lot of work. The enzyme according to the present invention can be advantageously recovered with ease because it is produced extracellularly. For example, in a liquid culture, the enzyme can be recovered by removing cells by filtration or centrifugation to obtain a filtrate or supernatant and then subjecting it to various types of chromatography. In a solid culture, after adding water to the cultured medium, the enzyme can be recovered in the same manner as in a liquid culture, namely by removing cells by filtration or centrifugation to obtain a filtrate or supernatant and then subjecting it to various types of chromatography.

Examples of methods for purifying cholesterol oxidase from a liquid culture include fractional precipitation, such as salting out with ammonium sulfate and solvent precipitation, and chromatography, such as ion-exchange chromatography, hydrophobic chromatography, gel-filtration chromatography, or the like. If necessary, desalting, for example by dialysis, can be carried out. The type and order of chromatography are not particularly restricted.

Use of Enzyme

The enzyme according the present invention has cholesterol oxidase activity. Therefore, the present invention provides reagents containing the enzyme according to the present invention, for measuring the concentration of 3β-sterols (particularly cholesterol). The concentration of 3β-sterols can be measured by contacting a sample with the enzyme of the present invention and measuring the degree of oxidation of the substrate, or the cholesterol oxidase activity. The sample can be isolated, for example, from mammals or from food products.

Cholesterol oxidase activity can be evaluated by measuring the amount of consumed oxygen using an oxygen electrode, or by measuring the amount of hydrogen peroxide generated during the oxidation reaction.

The present invention also provides a method for producing sterol derivatives. Sterol derivatives can be produced by contacting the enzyme of the present invention with 3β-sterols, for example, in an organic solvent, to recover oxidized 3β-sterols. Examples of sterol derivatives that can be produced include oxidized forms of 3β-sterols such as oxidized cholesterol, e.g., cholest-5-en-3-one and 6β-perhydroxycholest-4-en-3-one.

Cholesterol oxidase destroys the epithelium of mesogaster of insects to kill them (Purcell J. P. et al., Biochem. Biophy. Res. Comm., Vol. 196, No. 3, 1406-1413 (1993); U.S. Pat. No. 5,558,862, etc.). Accordingly, the present invention provides compositions containing the enzyme of the present invention, for exterminating pests. The term “compositions for exterminating pests” as used herein include agents for plant protection, agents for suppressing pests, insecticides, pesticides, or the like.

Compositions for exterminating pests according to the present invention can be prepared, for example, by adding the enzyme of the present invention to appropriate desired ingredients (e.g., surfactants, moisturizers, solid diluents, dispersing agents, UV stabilizers, and the like) to formulate appropriate dosage forms such as wettable powders, powders or flowable agents.

Furthermore, compositions for exterminating pests according to the present invention can include various pesticides, germicides, weed killers, plant growth controlling agents, synergists (including activity enhancing substances contained in a culture supernatant), inducing agents, plant nutrients, fertilizers or the like, as required and/or desired during preparation or spraying.

Extermination of pests can be generally carried out by spraying compositions for exterminating pests, diluted with diluents (e.g., water) or without dilution, onto plants damaged by pests or plants on which damages are anticipated.

Cholesterol oxidase acts on a substrate to yield hydrogen peroxide. Enzymes which can produce hydrogen peroxide have bleaching effect and can be added to detergents as a bleaching agent (WO89/09813; Japanese Patent Laid-open No. 505100/1991). Accordingly, the present invention provides detergent compositions containing the enzyme according to the present invention.

The detergent compositions according to the present invention can include the enzyme of the present invention alone or in combination with surfactants, builders and other auxiliaries (e.g., fluorescent whitening agents, foam boosters, sud controllers, softening agents, perfume).

The enzyme according to the present invention is characterized by its high reaction velocity even under a low concentration of a substrate. Furthermore, the enzyme according to the present invention has heat stability better than conventional natural cholesterol oxidases or heat stability equivalent to genetically engineered enzymes that are transformed by gene recombinant technology to increase heat stability. Accordingly, the enzyme according to the present invention is advantageously used for measuring the concentration, in particular low cholesterol concentrations, of 3β-sterols in body fluids or food products.

The enzyme according to the present invention is also characterized by its high reaction velocity in an organic solvent. Since most conversions of sterols by enzymes are carried out in an organic solvent, the converting enzymes need to be active in an organic solvent. Advantageously, the enzyme according to the present invention converts sterols with high efficiency in an organic solvent.

### EXAMPLES

The present invention is further illustrated by the following Examples that are not intended as a limitation of the invention.

### Example 1

### Purification of Enzyme

Pseudomonas strain ST-200 (FERM BP-6661) were cultured at 30° C. for 17 hours in 6 L of a medium containing 1% tryptone (Difco), 0.5% yeast extract (Difco) and 1% sodium chloride in a 10-L fermenter. The rate of stirring was 400 rpm and aeration was 12 L per minute.

The resulting culture fluid was centrifuged at 8,000×g for 15 minutes to obtain supernatant. The supernatant was subjected to salting out by adding ammonium sulfate to 70% saturation (4° C., overnight) and the resulting precipitate was recovered by centrifugation at 10,000×g for 30 minutes. The precipitate thus obtained was dissolved in a 10 mM tris-hydrochloric acid buffer solution (pH 8) and subjected to dialysis twice against the same buffer solution.

The dialyzed precipitate was then loaded on a DEAE cellulose DE 52 column and isocratic elution was carried out with a 10 mM tris-hydrochloric acid buffer solution (pH 8). Ammonium sulfate was added to an active fraction to the final concentration of 45% saturation, after which the resulting supernatant was recovered by centrifugation (7,000×g, 15 minutes). The activate fraction was loaded on a column filled with Butyl Toyopearl 650S equilibrated with a 10 mM tris-hydrochloric acid buffer solution (pH 8) containing ammonium sulfate at 45% saturation of and eluted with a linear gradient of ammonium sulfate down to 0 M. Fractions of the ammonium sulfate gradient from 10% saturation to 0 M, in which activity was located, were collected and subjected to salting out by adding ammonium sulfate to 80% saturation.

The resulting precipitate was dissolved in a 10 mM tris-HCl buffer solution (pH 8), then dialyzed twice against the same buffer solution. Fractionation was then carried out using a Sephadex G-100 column with a solution containing 10 mM tris-HCl buffer solution (pH 8), 50 mM sodium chloride and 5 mM sodium cholate. An active fraction was collected and dialyzed against a 10 mM tris-HCl buffer solution (pH 8) to obtain purified cholesterol oxidase.

The yield of the active sample thus obtained was 20%. Specific activity was 15.2 U/mg, which was 36 times that before purification. The molecular weight measured by SDS-polyacrylamide gel electrophoresis was 60 kDa. The enzyme was characterized to strongly oxidize cholesterol, β-sitosterol, β-cholestanol and the like, but not act on epicholesterol, and be strongly inhibited by silver nitrate or mercury chloride.

### Test Example 1

### Effect of pH and Temperature on Enzyme

The pH and temperature dependence and stability of the enzyme obtained in Example 1 were measured.

The pH and temperature dependence was measured by measuring oxygen consumption when cholesterol was used as a substrate. More specifically, the enzyme according to the present invention was added to a solution containing a 50 mM phosphate buffer solution (pH 7.0), 64 mM sodium cholate, 0.34% surfactant Triton-100 and 0.89 mM cholesterol to measure the oxygen consumption. The amount of oxygen was measured by a DO meter (YSI Model 53, Yellow Spring, Ohio, USA). Activity to oxidize 1 μmole of cholesterol per minute was defined as one unit of cholesterol oxidase activity. For pH dependence, activity at pH 7 was set to be 100%. For temperature dependence, activity at 60° C. was set to be 100%.

In order to study the pH and temperature stability, the enzyme was maintained at different pHs and temperatures, and the remaining activity was then measured by measuring hydrogen peroxide produced in the enzyme reaction at 30° C. at pH 7 using cholesterol as a substrate. More specifically, the measurement was carried out as follows.

To a solution containing the enzyme according to the present invention in an appropriate concentration were added (in each case to a final concentration) 50 mM phosphate buffer (pH 7.0), 64 mM sodium cholate, 0.34% surfactant TritonX-100, 1.4 mM aminoantipyrine, 21 mM phenol, 5 unit peroxidase derived from wasabi, Japanese horseradish (Toyobo), and 0.89 mM cholesterol; 3 ml of the solution so prepared was reacted at 30° C. for 5 minutes while measuring optical density at 500 nm. Activity to oxidize 1 μmole of cholesterol per minute was defined as one unit of cholesterol oxidase activity.

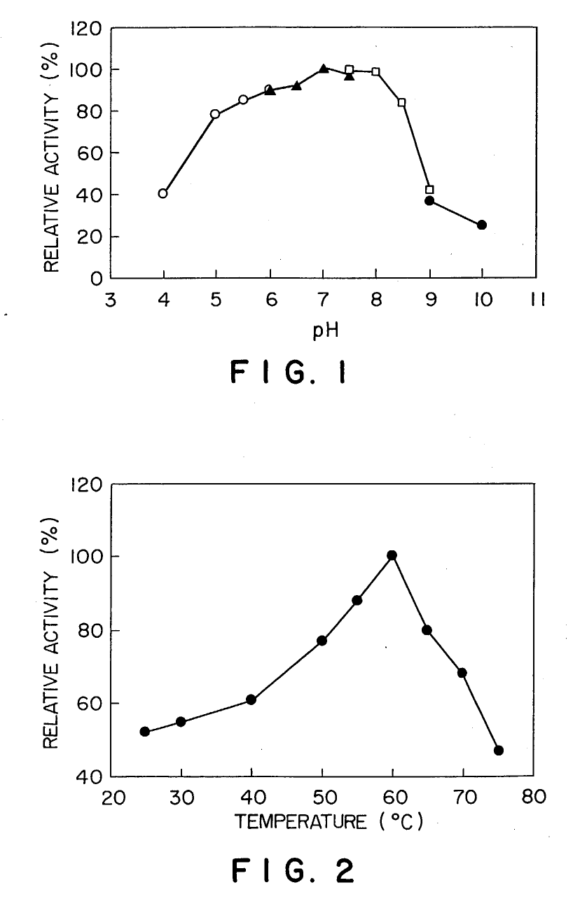

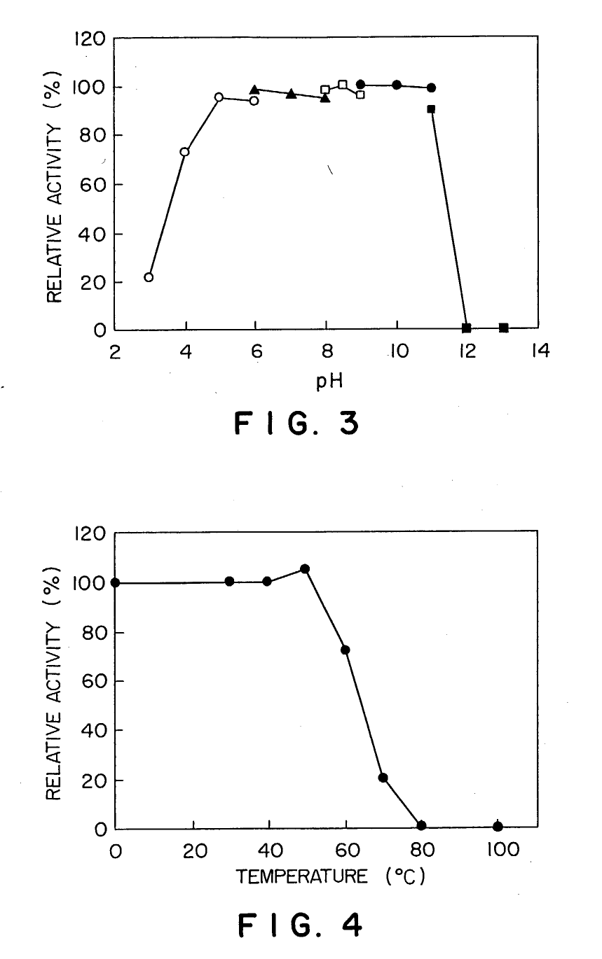

Results are shown in FIGS. 1 to 4. The enzyme according to the present invention showed strong activity at pHs between 5.0 and 8.5, and was stable at pHs between 4.0 and 11.0. The enzyme also showed strong activity at 50° C. to 60° C. and was stable at 4° C. to 55° C.

### Test Example 2

### Substrate Specificity of Enzyme

Substrate specificity of the enzyme obtained in Example 1 was studied. Enzyme activity was measured using various substrates, as described in Test Example 1 at 30° C. at pH 7. As shown by the results in Table 1, the enzyme strongly oxidized cholesterol, β-sitosterol and β-cholestanol, but did not act on epicholesterol, i.e., a 3α-hydroxysteroid.

TABLE 1
Substrate specify of cholesterol oxidase derived
from ST-200 strain

| | Relative |
|---|---|
| Substrate | activity (%) |
| Cholesterol (cholest-5-en-3β-ol) | 100 |
| β-sitosterol (sitost-5-en-3β-ol) | 84 |
| β-cholestanol (5-α-cholestan-5-en-3β-ol) | 69 |
| β-stigmasterol (Stigmast-5-en-3β-ol) | 59 |
| Pregnenolone (3β-Hydroxypregn-5-en-20-one) | 32 |
| Ergosterol (Ergosta-5,7,22-trien-3β-ol) | 20 |
| Dehyoepiandrosterone (3β-Hydroxyandrost-5-en- | |
| 17-one) | 16 |
| Epiandrosterone (5α-androstan-3β-ol-17-one) | 10 |
| Epicholesterol (Cholest-5-en-3α-ol) | 0 |

Enzyme activity with cholesterol was set to be 100.

### Test Example 3

### Effect of Metal Ions on Enzyme Activity

Effect of metal ions and the like on the activity of the enzyme obtained in Example 1 was studied. Enzyme activity was measured as described in Test Example 1 at 30° C. at pH 7 after adding various metal compounds, to a final concentration of 1 mm, to the reaction system. As shown by the results in Table 2, enzyme activity was strongly inhibited by silver nitrate or mercury chloride.

TABLE 2
Effect of metal ions and the like on cholesterol oxidase
derived from ST-200 strain

| Additives | Relative activity (%) |
|---|---|
| None | 100 |
| Calcium chloride | 106 |
| Magnesium chloride | 102 |
| Iron sulfate | 94 |
| Copper sulfate | 105 |
| Nickel chloride | 90 |
| Manganese chloride | 92 |
| Zinc chloride | 92 |
| Silver nitrate | 2 |
| Mercury chloride | 0 |
| EDTA | 88 |
| 8-hydroxyquinoline | 85 |
| α,α-dipyridyl | 98 |
| o-phenanthroline | 92 |
| Sodium azide | 95 |

The concentration of each additive was 1 mM. Activity without additive was set to be 100.

### Test Example 4

### Enzyme Kinetics

Michaelis constant (Kₘ) and maximum reaction velocity (Vₘₐₓ) of the enzyme obtained in Example 1 were studied in the presence of 0.03% and 0.3% surfactant Triton X-100. Enzyme activity was measured as described in Test Example 1 at 30° C. at pH 7. As shown by the results in Table 3, the cholesterol oxidase according to the present invention showed a smaller Kₘ and a larger Vₘₐₓ and the largest Vₘₐₓ/Kₘ ratio as compared to those derived from strains of Nocardiaerythropolis, Pseudomonas sp., Streptomyces sp., and Brevibacterium sp.

TABLE 3
Michaelis constant (Kₘ) and maximum reaction
velocity (Vₘₐₓ) of cholesterol oxidase

| | 0.03% Triton X-100 added | | | 0.03% Triton X-100 added | | |
|---|---|---|---|---|---|---|
| | | Vₘₐₓ | | | Vₘₐₓ | |
| Kₘ | (μmole | | Kₘ | (μmole | | |
| Origin | (μM) | min⁻¹mg⁻¹) | Vₘₐₓ/Kₘ | (μM) | min⁻¹mg⁻¹) | Vₘₐₓ/Kₘ |
| ST-200 | 4.04 | 13.1 | 3.2 | 52.2 | 12.1 | 0.23 |
| Nocardia- | 5.14 | 9.8 | 1.9 | 44.0 | 6.8 | 0.15 |
| erythropolis | | | | | | |
| Pseudomonas | 9.41 | 11.0 | 1.1 | 76.1 | 10.3 | 0.13 |
| sp. | | | | | | |
| Streptomyces | 16.3 | 15.8 | 0.9 | 160 | 15.3 | 0.01 |
| sp. | | | | | | |
| Brevibacterium | 63.3 | 12.0 | 0.2 | 925 | 11.3 | 0.01 |
| sp. | | | | | | |

Kₘ and Vₘₐₓ were obtained from Lineweaver-Burk plots with the reaction velocities measured in the presence of 0 to 1 mM cholesterol.

### Test Example 5

### Effect of Organic Solvents on Enzyme Activity

Effect of organic solvents on activity of the enzyme obtained in Example 1 was studied. Each organic solvent (50% by volume of reaction solution) was added at the time of measurement of activity. Enzyme activity was measured at 30° C. at pH 7 as described in Test Example 1. As shown by the results in Table 4, cholesterol oxidase of the present invention showed higher reaction velocity in benzene, toluene, paraxylene, propylbenzene, diphenylmethane and cyclooctane as compared to those derived from strains of Nocardiaerythropolis, Pseudomonas sp., Streptomyces sp., and Brevibacterium sp.

TABLE 4
Effect of organic solvents on activity of cholesterol
oxidase derived from ST-200 strain

| | | Relative activity | | | | |
|---|---|---|---|---|---|---|
| Organic | | | Nocardia | Pseudo- | Strepto- | Brevibacter- |
| solvents | LogP_{OW} | ST-200 | erythropolis | monas sp. | myces sp. | ium sp. |
| None | — | 1 | 1 | 1 | 1 | 1 |
| Chloroform | 1.9 | 0.2 | 0.3 | 0.1 | 0.1 | <0.1 |
| Benzene | 2.1 | 3.0 | 1.5 | 1.3 | 0.9 | 0.5 |
| Toluene | 2.6 | 3.5 | 1.7 | 1.4 | 1.2 | 0.5 |
| Paraxylene | 3.1 | 3.4 | 1.6 | 1.5 | 1.1 | 0.7 |
| Propylbenzene | 3.7 | 3.2 | 1.6 | 1.3 | 1.1 | 0.8 |
| Diphenylmethane | 4.2 | 3.1 | 1.6 | 1.5 | 1.0 | 0.8 |
| Cyclooctane | 4.5 | 1.4 | 1.1 | 0.8 | 0.4 | 0.2 |

Activity was measured at pH 7 at 30° C.

1. Cholesterol oxidase produced by ST-200 strain (FERM BP-6661):

2. An enzyme having the following properties:
(1) Function: acting on cholesterol to convert it to cholest-5-en-3-one; acting on cholest-5-en-3-one to convert it to 6β-perhydroxycholest-4-en-3-one;
(2) Substrate specificity: acts on 3β-sterols and not on 3α-hydroxysteroid;
(3) Optimum pH: 5.0 to 8.5; and
(4) Stable pH: 4 to 11.

3. An enzyme as claimed in claim 2 further having the following properties:
(5) Optimum temperature: approximately 60° C.;
(6) Stable temperature: 4° C. to 55° C.;
(7) Reaction velocity being increased in the presence of organic solvents having 2.1 to 4.5 of a common logarithm of partition constant P_{ow} of a given substance between water and n-octanol (log P_{ow});
(8) Molecular weight: approximately 60 kDa as measured by SDS-PAGE; and
(9) Enzyme activity being inhibited by silver nitrate and mercury chloride when cholesterol is used as a substrate.

4. An enzyme as claimed in claim 2 wherein a ratio of a maximum reaction velocity (Vₘₐₓ: μmole·min⁻¹·mg⁻¹) to Michaelis constant (Kₘ: μM), Vₘₐₓ/Kₘ, is 0.20 or more in the presence of 0.3% surfactant Triton X-100, or the Vₘₐₓ/Kₘ is 3.0 or more in the presence of 0.03% Triton X-100.

5. An enzyme as claimed in claim 2 which is produced by a cyclohexane-resistant microorganism.

6. An enzyme as claimed in claim 2 which is produced by a cyclohexane-resistant microorganism of genus Pseudomonas.

7. An enzyme as claimed in claim 2 which is produced by ST-200 strain (FERM BP-6661).

8. A method for measuring 3β-sterols concentration, comprising the steps of contacting a sample with the enzyme as claimed in any one of claims 1 to 7 and measuring the cholesterol oxidase activity.

9. A method for measuring 3β-sterols concentration as claimed in claim 8 wherein the sample is isolated from mammals or from food products.

10. A reagent for the measurement of 3β-sterols concentration, comprising the enzyme as claimed in any one of claims 1 to 7.

11. A composition for the extermination of pests, comprising the enzyme as claimed in any one of claims 1 to 7.

12. A detergent composition, comprising the enzyme as claimed in any one of claims 1 to 7.

13. A method for producing cholesterol derivatives, comprising the steps of contacting 3β-sterols with the enzyme as claimed in any one of claims 1 to 7 and recovering the corresponding sterol derivative.

14. A method as claimed as claimed in claim 13 wherein the enzyme is contacted with 3β-sterols in an organic solvent.
